(19) United States
(12) Patent Application Publication
(10) Pub. No.: US 2009/0076373 A1
(43) Pub. Date: Mar. 19, 2009

(54) Medical imaging facility, in particular for producing image recordings in the context of a treatment of cardiac arrhythmias, and associated method

(75) Inventor: Michael Maschke, Lonnerstadt (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 12/283,417

(22) Filed: Sep. 11, 2008

(30) Foreign Application Priority Data
Sep. 13, 2007 (DE) 10 2007 043 731.7

(51) Int. Cl. A61B 5/055

(52) U.S. Cl. 600/410; 382/131

(57) ABSTRACT

The invention relates to a medical imaging facility, in particular for producing image recordings in the context of a treatment of cardiac arrhythmias, the medical imaging facility being configured as an integrated facility with at least one computed tomography device configured as a C-arm computed tomography device and configured to produce soft-tissue recordings and at least one image recording element based on intravascular magnetic resonance imaging, the medical imaging facility having at least one common control facility for both image recording methods, being configured for the automatic evaluation and display of at least one image recording of at least one computed tomography facility and/or at least one image recording element based on intravascular magnetic resonance imaging and/or for the display of identified scar tissue and/or regions of impaired and/or increased electrophysiological activity by means of at least one program means in real time.

### CROSS REFERENCE TO RELATED APPLICATIONS

This application claims priority of German application No. 10 2007 043 731.7 filed Sep. 13, 2007, which is incorporated by reference herein in its entirety.

### FIELD OF THE INVENTION

The invention relates to a medical imaging facility, in particular for producing image recordings in the context of a treatment of cardiac arrhythmias, and associated method.

### BACKGROUND OF THE INVENTION

Cardiac arrhythmias are disruptions of the normal heartbeat sequence, whose causes can be found in disturbances of the formation or conduction of stimuli in the cardiac muscle.

One example of a cardiac arrhythmia is what is known as bradycardia, with which there is a drop in heart rate. For many years cardiac pacemakers have been used to treat bradycardia, these being intended to restore a normal sinus rhythm.

Other cardiac arrhythmias are tachycardial problems such as for example atrial fibrillation. With this type of cardiac arrhythmia interference with the conduction of impulses in the heart causes the atrium to be stimulated at a high frequency. In the case of ventricular tachycardias interference means that instead of a complete contraction of the cardiac muscle there is incomplete contraction, with the result that the heart pumps inadequately. Therapeutic measures for the treatment of tachycardias are a continuous drug regime or even heart operations, in which the impulse conducting tissue in certain parts of the heart is separated. This has the disadvantage of involving a relatively high level of risk for the patient.

For this reason minimally invasive therapy methods have increasingly been used in recent times to combat cardiac arrhythmias. In these an ablation catheter is inserted into the region of the heart by way of venous access, to destroy the defective conduction paths there, for example by applying energy to “burn” them. Successful implementation of such a therapy requires the interfering impulse conducting paths and/or points to be known and to be located accurately by the catheter. This method is deployed for example for supraventricular tachycardias and also increasingly in the field of ventricular tachycardias, particularly given that drug treatment with antiarrhythmic agents has only a low success rate and on the other hand implantable defibrillators have unpleasant side effects for the patient. However it remains difficult to identify and locate the correct points for the ablation during the intervention.

The use of an angiographic x-ray unit and a device for recording the intracardiac electrocardiogram in conjunction with a device for “cauterizing”, in other words for ablating, the tissue region with impaired impulse conduction, is known for the field of minimally invasive diagnosis and therapy. This diagnosis and therapy method is based on the determination of electrophysiological potentials in the heart. The measurement of electrophysiological potentials to determine ablation locations is generally referred to as mapping. The publications DE 44 36 828 C1 and U.S. Pat. No. 5,365,926 are examples of this prior art.

It is known from U.S. Pat. No. 6,556,695 B1 that a device can be used to assist with the diagnosis and high-frequency ablation of tissue with impaired impulse conduction as well as mapping, with which before the actual procedure three-dimensional image recordings are produced, which are overlaid with image recordings produced during therapy using an intracardiac ultrasound catheter. However the ultrasound catheter only supplies two-dimensional recordings, so that only limited image information with low resolution is available during the procedure, since the high-resolution recordings with three-dimensional data do not show the current situation during therapy.

The electrophysiological potentials do not allow actual tissue display, in particular any display of the scar tissue resulting in the heart for example in the context of a cardiac infarction. However ventricular tachycardias for example originate from what are known as reentrant circuits, which generally occur at and in the outer edges of the electrically inactive myocardial scar tissue, so the lack or inadequacy of the tissue display is problematic for the therapy.

### SUMMARY OF THE INVENTION

The object of the invention is therefore to specify a medical imaging facility that is improved in this respect, in particular for producing image recordings in the context of a treatment of cardiac arrhythmias, as well as an associated method.

To achieve this object a medical imaging facility of said type is provided, which is characterized in that it is configured as an integrated facility with at least one computed tomography device and at least one imaging element based on intravascular magnetic resonance imaging.

The medical imaging facility, which is preferably an imaging facility which is configured to treat cardiac arrhythmias, thus has means for producing computed tomography recordings and for producing intravascular magnetic resonance recordings in an integrated form.

This has the advantage that it is possible to produce both computed tomography recordings and intravascular magnetic resonance recordings using a single system, in other words without having to move the patient and carry out a new registration. Also an intravascular facility is used for magnetic resonance imaging, so there is no need for an expensive separate magnetic resonance tube or facility.

Intravascular magnetic resonance imaging (abbreviated to IVMRI) has the advantage that cardiac infarction scar tissue for example can be identified simply from the data obtained therewith.

The IVMRI subsystem can thus be used, optionally with the additional use of magnetic resonance contrast agents, to identify infarction scars and regions of impaired and increased electrophysiological activity. In this process IVMRI initially provides functional imaging.

The computed tomography device makes it possible in a parallel manner, in other words in particular in the context of a simultaneous recording, to display the relevant anatomy of the heart, in particular by producing what are known as soft tissue recordings. The computed tomography recordings of the integrated imaging facility can be recorded with and without the administration of x-ray contrast agent. It is also possible to produce recordings in the context of combined methods in respect of the use of contrast agents, in other words to produce recordings first of all with and then without contrast agent and then to overlay these or subtract them or otherwise process them together.

The medical imaging facility can be configured as a hybrid system. It is thus an imaging facility for treating cardiac arrhythmias, etc., which from the outset forms a combined system for computed tomography recordings and intravascular magnetic resonance recordings. This means that for example a common control facility is provided to produce the recordings or where there are a number of control facilities these are configured to record images in a coordinated manner. An appropriate common data bus can be used to organize image recording, including the pre-processing and post-processing of the images and their subsequent display, in a standard manner for both image recording methods. The image system of such a medical imaging facility is thus configured specifically for recording images both for computed tomography recordings and also intravascular magnetic resonance recordings.

At least one computed tomography device can be a C-arm computed tomography device. In particular specific cardiac C-arm computed tomography devices can be part of the medical imaging facility according to the invention. One example is the DynaCT (registered trademark) from Siemens AG.

The medical imaging facility can have at least one control facility, in particular a common control facility for both imaging methods. Image recording is therefore preferably controlled by a single control facility both for computed tomography recordings and also for intravascular magnetic resonance recordings. Naturally in certain exemplary embodiments a number of control facilities can also be present, in particular control facilities on the one hand for the computed tomography device and on the other hand for recording images by means of intravascular magnetic resonance imaging. However a common control facility allows particularly simple operation.

The control facility can be configured for the automatic evaluation of at least one image recording of the computed tomography device and/or at least one image recording element based on intravascular magnetic resonance imaging and/or for the identification of scar tissue, in particular cardiac infarction scar tissue, and/or regions of impaired and/or increased electrophysiological activity, by means of at least one program means. The control facility, which can for example be a console or computer facility or similar, thus has a storage region for a program means or program package or similar. Additionally or alternatively the control facility can have access to such a program means or package, it being possible for the automatic evaluation by the program means to take place optionally with the additional assistance of operator inputs. The program means is preferably able to evaluate the IVMRI or computed tomography recordings, in other words for example to determine and identify the scar tissue that is present for example after a cardiac infarction or tissue regions, in which there is impaired impulse conduction. Image processing algorithms are preferably used for this, for example edge recognition or recognition of deviating structures in certain image regions or pattern recognition. A comparison with data from at least one database can also be carried out to identify corresponding tissue regions for the treatment of cardiac arrhythmias.

The control facility can also be configured for the display of at least one image recording of at least one computed tomography device and/or at least one image recording element based on intravascular magnetic resonance imaging and/or for the display of identified scar tissue and/or regions of impaired and/or increased electrophysiological activity, by means of at least one program means. Such a display for example of images of the two imaging methods can be shown for example on a screen or monitor, which is assigned to a control facility. Displays on a monitor wall and such like are also possible. If the regions of impaired or increased electrophysiological activity or scar tissue are displayed, it is then possible, for example with a parallel implementation of an ablation of the impaired tissue to treat the cardiac arrhythmia, for the ablation process to be checked by an operator or by an image comparison or similar image processing method carried out automatically by a control facility or computer facility.

The control facility is then advantageously configured to display at least one image recording of at least one computed tomography device and/or at least one image recording element based on intravascular magnetic resonance imaging and/or of identified scar tissue and/or regions of impaired and/or increased electrophysiological activity in real time. During a catheter procedure to ablate tissue for example the real-time display allows direct, image-assisted observation based on the image recordings of the computed tomography device and intravascular magnetic resonance imaging. It is thus possible for an ablation to be guided automatically or by an operator.

At least one image recording element based on intravascular magnetic resonance imaging can be integrated, at least partially, into a catheter.

Integration into a catheter is advantageously such that for example one IVMRI sensor, optionally also a number of IVMRI sensors, are arranged on or in a catheter, which is in some instance inserted anyway, for example in the context of treatment of the cardiac arrhythmias, into the vascular system of the patient. An example of this is an ablation catheter, into which an IVMRI catheter can be integrated.

The medical imaging facility can therefore have at least one ablation catheter with at least one ablation means, in particular an ablation catheter with at least one integrated image recording element based on intravascular magnetic resonance imaging. In this instance the catheter therefore serves not only to produce the intravascular magnetic resonance recordings but also at the same time to ablate tissue with impaired impulse conducting behavior. Such an ablation catheter can have different ablation means, for example a high-frequency coil, ultrasound generation means or ablation means based on cold and/or heat energy.

The medical imaging facility can also be configured, in particular by means of a control facility, for a three-dimensional image recording, to rotate at least one image recording element based on intravascular magnetic resonance imaging and at the same time to withdraw and/or advance the catheter with at least one integrated image recording element in a blood vessel. For example an IVMRI sensor or another IVMRI element for generating layer images can be rotated and at the same time withdrawn or advanced, it being possible for this withdrawal or advance, like the rotation, to be effected fully automatically by a control facility or to be implemented (additionally) with operator assistance. Three-dimensional recordings can be produced in this manner. This allows anatomical assignment for example of infarction tissue or other tissue with impaired impulse conduction.

At least one position sensor element can be provided in the region of a tip of the catheter with at least one integrated image recording element based on intravascular magnetic resonance imaging. Such a position sensor facility allows infarction tissue and other tissue with impaired impulse conduction to be identified and spatially assigned, so that an ablation can be performed accurately in this region. Position sensors can also be used for example in conjunction with IVMRI imaging elements, to prevent motion artifacts in a non-precise three-dimensional display.

At least one position sensor element can be an electromagnetic or ultrasound-based position sensor element. Naturally combinations of different sensor elements can be used, for example a number of ultrasound-based sensors combined with a number of electromagnetically based sensors, to allow failsafe position identification at all times with redundancy for example.

At least one position sensor element in the region of the tip of the catheter can be a transmitter, to which at least one receiver is assigned outside the body of the patient, and/or at least one position sensor element in the region of the tip of the catheter can be a receiver, to which at least one transmitter is assigned outside the body of the patient. The electromagnetic transmitters or alternatively the electromagnetic receivers can then be arranged in the catheter for example. The corresponding receivers or in the second instance the transmitters are then arranged outside the body. In this process at least one transmitter is generally assigned to one receiver or conversely one receiver to one transmitter, to allow spatial location. In certain instances however it may also be sufficient to assign two transmit devices to one receiver or vice versa, if for example the angle relationships are known and do not change.

An increased number of transmit and receive units in space allows the location accuracy to be improved. It should however be noted that the use of a number of transmitters or receivers is also associated with a corresponding increase in computation outlay for example for position calculation on the part of a control facility of the medical imaging facility.

Furthermore at least one transmitter can be configured to transmit in all three spatial directions and/or at least one receiver can be configured to receive in all three spatial directions. This allows location in three-dimensional space in a simple manner.

At least one computed tomography device of the medical imaging facility can be configured to produce two-dimensional and/or three-dimensional images, in particular of the anatomy of at least one sub-region of the heart of a patient. A three-dimensional display is preferable here, in particular of soft-tissue recordings. The relevant anatomy can be displayed and the corresponding recordings can be produced with or without x-ray contrast agents. It is also possible to produce recordings both with and without the use of contrast agent and overlay or subtract these from one another correspondingly or process them together in any other manner.

The medical imaging facility, in particular a control facility, can also be configured to register and/or merge image recordings of at least one computed tomography device with image recordings of at least one imaging recording element based on intravascular magnetic resonance imaging. This allows identification of ventricular scar tissue for example in the x-ray images produced correspondingly with IVMRI data etc.

The medical imaging facility, in particular a control facility, can be configured to guide a catheter with at least one integrated image recording means based on intravascular magnetic resonance imaging as a function of x-ray recordings of at least one computed tomography device and/or recordings of at least one image recording element based on intravascular magnetic resonance imaging. Merged x-ray recordings and computed tomography recordings in particular can advantageously be used for this purpose, containing x-ray data and additional data from intravascular magnetic resonance imaging. Such recordings containing IVMRI data, showing the tissue with impaired impulse conduction or similar, and containing x-ray data, which shows the anatomy, allow particularly reliable and precise guidance, for example of an ablation catheter.

The computed tomography device can have a buckling arm robot, on which at least one x-ray emitter and/or one x-ray radiation detector are arranged. Such robot-type facilities allow easier access to the patient and additional possibilities for x-ray projections. Preferably at least four, or even better six, degrees of freedom of movement should be provided.

The medical imaging facility can also have a catheter tracking system. In this instance the position and/or path through the vascular system of a catheter which has image recording elements, as described above, for IVMRI imaging and ablation means or similar, can be tracked by way of a corresponding system. A catheter tracking system allows the tip of an ablation catheter to be detected for example, without x-ray radiation having to be emitted continuously. Such a system is described for example in U.S. Pat. No. 5,042,486.

The medical imaging facility can also have at least one image recording element for producing, in particular three-dimensional, ultrasound recordings, in particular in real time. In this instance an ultrasound facility is provided in addition to the IVMRI facility, so that further data is available for example for the supplementary guidance of an ablation procedure. The ultrasound guidance of medical instruments for a hybrid system of computed tomography and IVMRI is a completely new approach. This is particularly so in conjunction with C-arm computed tomography devices, which have the advantage of significantly improved patient access.

The connections of the medical imaging facility, in particular connections for at least one physiological sensor and/or catheter, can be decoupled from any mains voltage by way of metallic isolation, in particular by means of optical decoupling. Such optical decoupling serves to exclude any danger to the patient.

The medical imaging facility can also have at least one movement detection sensor and can optionally be configured to take the sensor data into account when reconstructing at least one image recording. This allows possible patient movement and resulting artifacts to be identified at the time of the examination or treatment. Patient movement can be taken into account directly with the aid of the sensor data during reconstruction of the recordings, thereby preventing the occurrence of motion artifacts in the finished reconstructed images.

At least one pressure and/or temperature sensor can be arranged in the region of the tip of a catheter with at least one image recording element based on intravascular magnetic resonance imaging and/or an ablation catheter. Naturally further sensors can likewise be provided to record physiological, physical and/or chemical data at such a catheter. However the monitoring of temperature and/or pressure in the organ or vessel is advantageous, particularly in the context of an ablation.

The invention also relates to a method for producing and/or evaluating at least one image recording of a medical imaging facility, in particular a medical imaging facility as claimed in one of the preceding claims, according to which at least one image recording is produced by means of a medical imaging facility configured as an integrated facility with at least one computed tomography device and at least one image recording element based on intravascular magnetic resonance imaging and/or according to which at least one produced image recording of the computed tomography device and/or at least one image recording element based on intravascular magnetic resonance imaging is evaluated automatically by a control facility of the medical imaging facility and/or is registered and/or merged with at least one further image recording.

With the method computed tomography images and IVMRI images are therefore recorded using an integrated facility, preferably in the form of a hybrid system, and optionally automatically evaluated and further processed, for example in the form of a registration or merging, in particular with further recordings of the medical imaging facility.

This image recording and/or image evaluation method, which per se only relates to the recording and/or evaluation of physical measured data and can also be carried out by a technician or natural scientist, can be part of a method for treating ventricular tachycardias or other tachycardias as well as ventricular flutter and fibrillation and the ablation of tumors and metastases. For example a patient can be positioned on a treatment table for treatment of a ventricular tachycardia. A magnetic resonance contrast agent such as gadolinium can then optionally be injected. Administration of a different contrast agent is also possible.

An IVMRI examination can then be carried out, for example after insertion of a corresponding IVMRI catheter. A two-dimensional or three-dimensional x-ray examination can then take place in addition, in particular in parallel, using a computed tomography device of the medical imaging facility. In particular processing can take place in respect of soft-tissue identification, optionally with synchronization by means of an electrocardiogram.

The IVMRI recordings and the x-ray recordings of the computed tomography device are advantageously merged in real time, in order to be able to assign scar tissue and the anatomy of a ventricle for example. A mapping system can also be used for electrophysiological imaging or the pictorial display of electrical potentials. Such a display can be overlaid on a display containing IVMRI data, x-ray data or the merged IVMRI data and x-ray data.

For the ablation an ablation catheter can be inserted, for example into the left ventricle, by way of venous access, for example the aorta. The regions in the ventricle, which develop unwanted electrophysiological activity, are then cauterized or obliterated with the aid of the ablation catheter. The ablation catheter is guided on the basis of the merged IVMRI and x-ray data, optionally also the data of the individual image recording systems. Ablation can take place by means of high-frequency radiation, ultrasound, cold and/or heat energy. This can be followed by a new IVMRI and/or mapping examination to check the success of the ablation. If the ablation has been successful, the patient can be moved. Otherwise a new ablation activity is required.

It is thus possible with the inventive hybrid system and/or integrated medical facility with the option of producing computed tomography recordings and IVMRI recordings to improve the medical workflow with more reliable and faster ablation of tissue parts with a lower patient risk.

### BRIEF DESCRIPTION OF THE DRAWINGS

Further advantages, features and details of the invention will emerge from the exemplary embodiments and drawings which follow, in which drawings:

FIG. 1 shows an inventive medical imaging facility,

FIG. 2 shows a catheter with an integrated IVMRI image recording element,

FIG. 3 shows a display of the ablation of tissue parts, based on data of an inventive medical imaging facility and

FIG. 4 shows a display of the read-out of measured data of an inventive medical imaging facility.

### DETAILED DESCRIPTION OF THE INVENTION

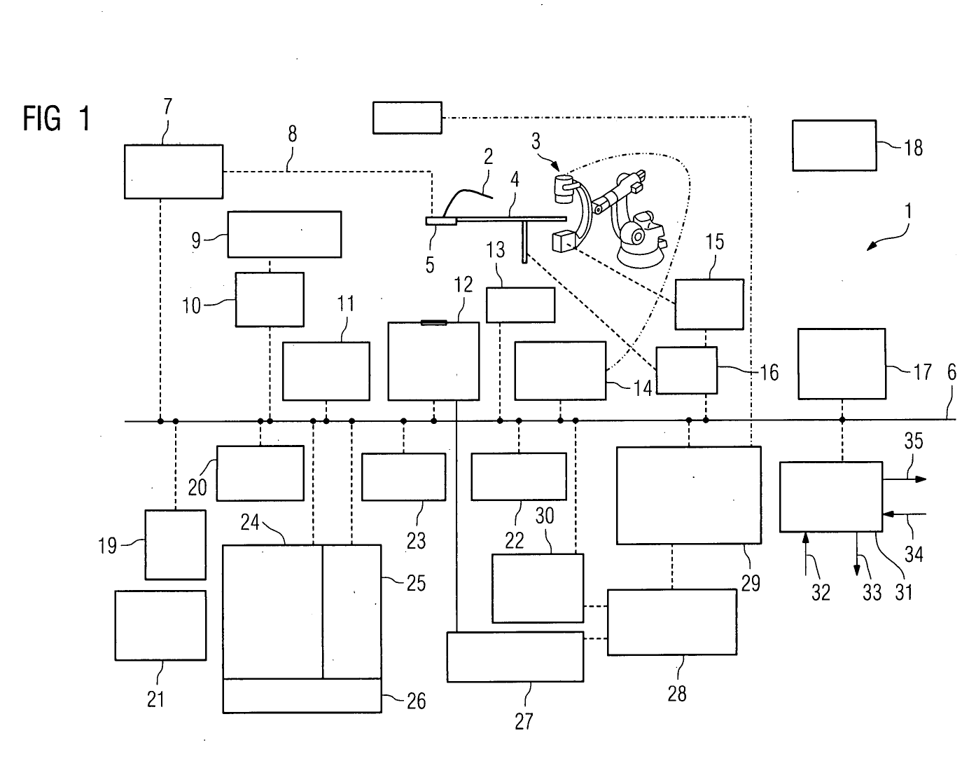

FIG. 1 shows an inventive medical imaging facility 1, configured as a hybrid system with an IVMRI catheter 2 with corresponding IVMRI image recording elements and a computed tomography device 3, here in the form of a buckling arm robot with at least 4 degrees of movement or degrees of freedom of movement.

A patient support facility 4 is also provided. An operating and interface facility 5 is used to record and forward the IVMRI date and allows operation of the IVMRI image recording elements in proximity to the table.

An IVMRI preprocessing and control unit 7 is connected to a data bus 6.

A connection 8 for an ultrasound catheter is also provided on the patient support facility 4 to produce ultrasound images. A signal interface 9 for the ultrasound data is connected to a preprocessing unit 10 for ultrasound data, which in turn is connected to the data bus 6.

The catheters of the system are provided with position sensors, to which a preprocessing unit 11 is assigned.

The patient support facility 4 also has connections (not shown in more detail here) for physiological sensors, for which a physiological signal processing unit 12 is provided, which for example processes the data of an electrocardiogram, respiration data and blood pressure data and temperature data as well as further physiological, physical and medical data obtained using the system.

The medical imaging facility 1 also has an ablation device 13 for ablating tissue with impaired impulse conduction. A further processing unit 14 is provided for preprocessing the x-ray images of the computed tomography device 3, the computed tomography device 3 being connected for radiation generation to a high-voltage generator 15, which is connected to a system controller 16.

An image data storage unit 17 is also provided at the data bus 6.

A defibrillator or cardiac pacemaker can also be contained in the system according to the box 18.

An image processing unit 19 for the IVMRI data and an image processing unit 20 for ultrasound data, such as data from an element for recording intravascular ultrasound recordings (IVUS), are also connected to the data bus 6. A power supply unit 21 is also provided.

The x-ray images of the computed tomography device 3 are further processed in the image processing unit 22 for x-ray images; the data from the position sensors is further processed in the image processing unit 23 for position sensors.

The medical imaging facility 1 also has a display unit 24 with a unit 25 for user input and output and a three-dimensional display controller 26. Ultrasound data, IVMRI data and images, position sensor images and the x-ray images of the computed tomography device 3 can be displayed on the display unit 24.

Synchronization based on an electrocardiogram for a soft-tissue processor is also provided in the system according to the box 27. The data to be synchronized accordingly originates from the image reconstruction unit 28, which also represents the soft-tissue processor. The merged or registered images are produced with the aid of the image merging, registration and reconstruction unit 29, which can be used among other things to carry out a segmentation or automatic segmentation of the image data. The medical imaging facility 1 also has a calibration unit 30.

An interface 31 for patient data and image data preferably according to the Digital Imaging and Communications in Medicine standard (DICOM standard) is also provided at the data bus 6. Data, for example from prior computed tomography or magnetic resonance recordings, can be recorded and retrieved by way of the interface 31, as shown here by the arrows 32 and 33. The interface 31 is also connected to an information system of an associated hospital or clinic, etc., as shown by the arrows 34 and 35, which symbolize a corresponding data exchange.

It is therefore possible with the inventive medical imaging facility 1, for example for the treatment of cardiac arrhythmias, to record computed tomography data and IVMRI data and optionally also further data using a single integrated system, in order thus to obtain image data, which shows the anatomy and at the same time functional data for example as tissue with impaired impulse conduction in a region to be ablated in an optimum manner.

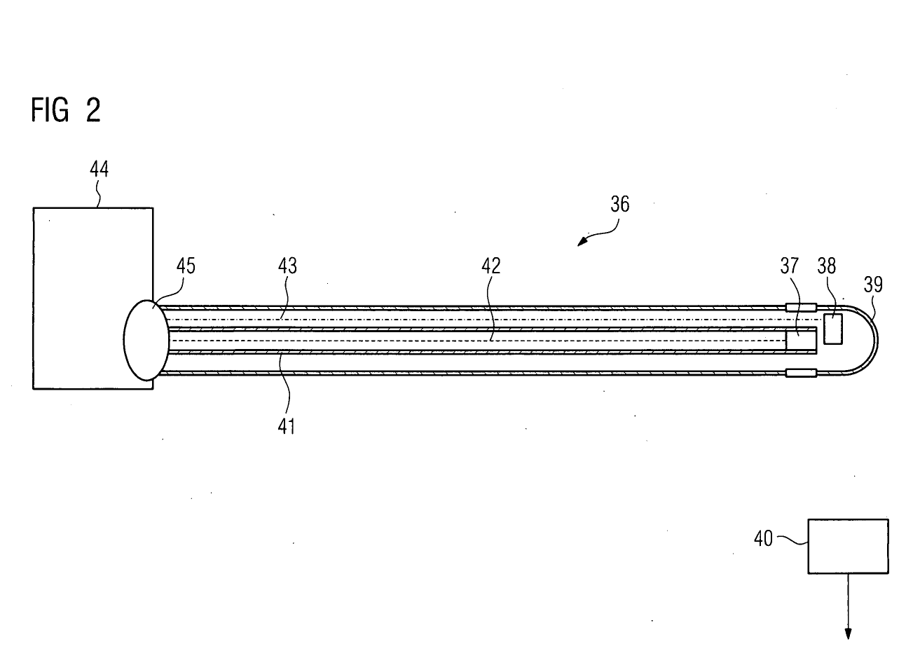

FIG. 2 shows a catheter 36 with an integrated IVMRI image recording element for use with an inventive method and/or in an inventive medical imaging facility.

In the region of its tip 39 the catheter 36 has an IVMRI sensor 37 with an assigned IVMRI-transparent window in the catheter sleeve as well as magnetic sensors 38 for position determination. The tip 39 is configured in a rounded manner.

To evaluate the position data an interface 40 with a position identification unit (not shown in more detail here) is provided.

The catheter 36 is configured with a lumen 41 for the IVMRI sensor 37, the IVMRI signal lines 42 and a drive shaft. The magnetic sensors 38 forming antennas are connected by way of signal lines 43 to a signal and mechanical interface 44 with an associated drive unit for the components of the catheter 36. The connection is realized with the aid of a coupling device 45.

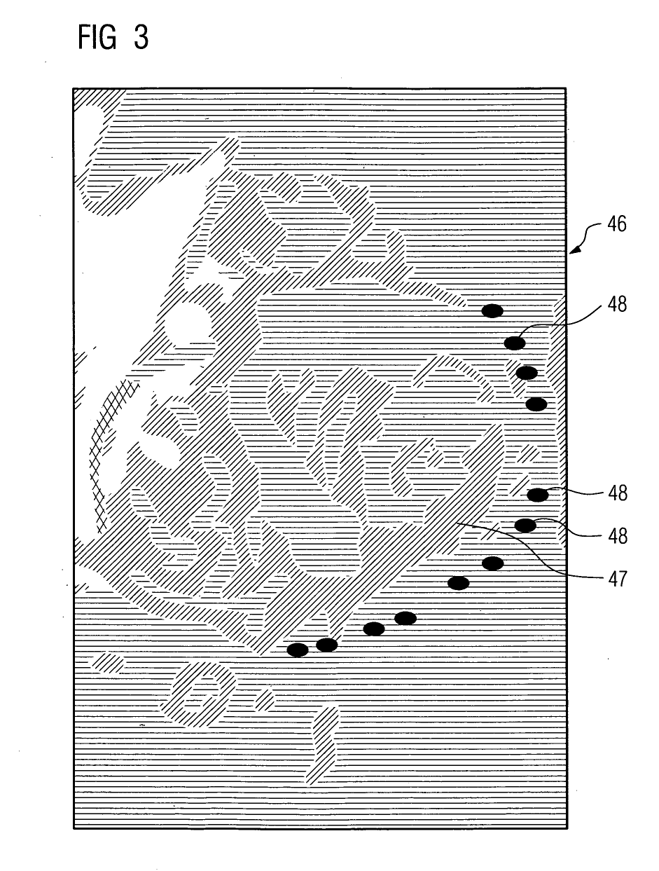

FIG. 3 shows a display 46 of the ablation of tissue parts based on data from an inventive medical imaging facility. The display 46 shows scar tissue 47 and ablation points 48 for a possible tissue ablation. The display 46 is shown on a screen (not shown in more detail here) of an inventive medical imaging facility, the image data advantageously being displayed in real time, in order thus to allow optimal guidance of the ablation or an optimal accompanying image display for a treatment, in particular of cardiac arrhythmias. With the inventive medical imaging facility it is possible to carry out a more reliable and faster ablation of tissue with impaired impulse conduction with much less risk to the patient.

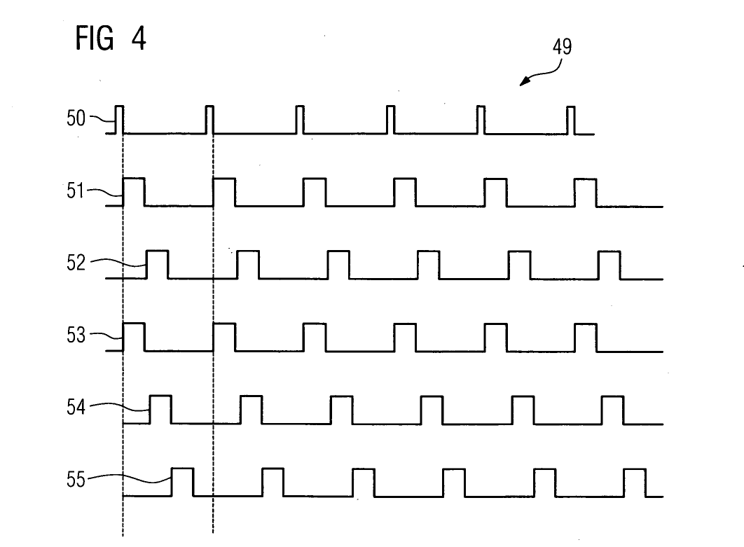

Finally FIG. 4 shows a display 49 of the read-out of measured data at an inventive medical imaging facility. Here the various sensors are temporally offset and are activated and read out in a timed manner for the synchronized read-out. The upper curve 50 here represents the system clock. The x-ray radiation is activated according to the curve 51 as a function of the end of the pulses of the system clock system according to the curve 50. The x-ray detector is then read out according to the curve 52.

Magnetic location is active according to the curve 53 parallel to the x-ray radiation.

The IVMRI data is read out according to the curve 54 at the same time as the x-ray detector is read out according to the curve 52. The data of the electrocardiogram and respiration data is read out according to the curve 54 following the reading out of the IVMRI data and the data from the x-ray detector according to the curves 52 and 54.

Data from an electrocardiogram or respiration data is then read out according to the curve 55.

1.-19. (canceled)

20. An integrated hybrid medical imaging system, comprising:
a computed tomography device that records a tomographic image of a patient;
an intravascular magnetic resonance imaging recording element that recodes an intravascular magnetic resonance image of the patient; and
a common control device that automatically evaluates the tomographic image and the intravascular magnetic resonance image.

21. The medical imaging system as claimed in claim 20, wherein the intravascular magnetic resonance imaging recording element is integrated in a catheter.

22. The medical imaging system as claimed in claim 21, wherein the catheter is an ablation catheter.

23. The medical imaging system as claimed in claim 21, wherein the control device rotates the intravascular magnetic resonance imaging recording element and simultaneously withdraws or advances the catheter in a blood vessel.

24. The medical imaging system as claimed in claim 21, wherein a position sensor element is provided in a region of a tip of the catheter.

25. The medical imaging system as claimed in claim 24, wherein the position sensor element is an electromagnetic position sensor element or an ultrasound-based position sensor element.

26. The medical imaging system as claimed in claim 24, wherein the position sensor element is a transmitter that transmits data in three spatial directions or a receiver that receives data in three spatial directions.

27. The medical imaging system as claimed in claim 21, wherein the control device guides the catheter based on the tomographic image or the intravascular magnetic resonance image.

28. The medical imaging system as claimed in claim 21, further comprising a catheter tracking system.

29. The medical imaging system as claimed in claim 21, wherein a connection between the medical imaging system and a physiological sensor or the catheter is decoupled from a voltage by a metallic isolation.

30. The medical imaging system as claimed in claim 20, wherein the tomographic image is a two-dimensional or a three-dimensional image of an anatomy of a sub-region of a heart of the patient.

31. The medical imaging system as claimed in claim 20, wherein the control device registers or merges the tomographic image with the intravascular magnetic resonance image.

32. The medical imaging system as claimed in claim 20, wherein the computed tomography device comprises a buckling arm robot on which an x-ray emitter or an x-ray radiation detector is arranged.

33. The medical imaging system as claimed in claim 32, wherein the buckling arm robot moves in four or six degrees of freedom.

34. The medical imaging system as claimed in claim 20, further comprising an ultrasound image recording element for recording an ultrasound image of the patient in real time.

35. The medical imaging system as claimed in claim 20, further comprising a movement detection sensor.

36. The medical imaging system as claimed in claim 20, wherein a pressure or a temperature sensor is arranged in a region of a tip of the catheter.

37. The medical imaging system as claimed in claim 20, wherein medical imaging system is used in a treatment of cardiac arrhythmia of the patient.

38. The medical imaging system as claimed in claim 20, wherein the control device evaluates and displays an identified scar tissue, a region of impaired, or an increased electrophysiological activity of the patient in real time.
